(19) United States
(12) Patent Application Publication
(10) Pub. No.: US 2023/0190527 A1
(43) Pub. Date: Jun. 22, 2023

(54) SURGICAL INSTRUMENT FOR GONIOTOMY PROCEDURE

(71) Applicant: RAICO International, LLC, Willowbrook, IL (US)

(72) Inventors: Alejandro Espaillat, Atlantis, FL (US); Ravi Nallakrishnan, Willowbrook, IL (US)

(21) Appl. No.: 17/927,408

(22) PCT Filed: May 26, 2021
(86) PCT No.: PCT/US2021/034258

§ 371 Date: Nov. 23, 2022

Related U.S. Application Data
(60) Provisional application No. 63/029,818, filed on May 26, 2020.

(51) Int. Cl. A61F 9/007

(52) U.S. Cl. CPC A61F 9/00772; A61B 2217/007; A61B 17/22004

(57) ABSTRACT

A goniotomy surgical instrument (10) includes a hand grip portion (12) having an elongated configuration with proximal and distal ends. The instrument (10) includes a tip portion (14) extending from the distal end of the hand grip portion (12) and includes a shank (16) extending from the hand grip portion (12). The tip portion (14) includes a V-shaped distal end having first and second legs (18, 20). The first leg (18) extends at a first angle (α) from the shank (16) of the tip portion (14). The second leg (20) extends at a second angle (β) from the first leg (18). The shank (16) and the first and second legs (18, 20) are substantially coplanar.

### PRIORITY

This application claims priority of U.S. Provisional Patent Application No. 63/029,818, filed May 26, 2020, the entire contents of which is incorporated herein by reference.

### FIELD OF THE INVENTION

The present invention relates to a micro-surgical instrument for performing ophthalmological procedures for treatment of eye diseases, such as glaucoma, and more particularly to a goniotomy surgical instrument having a tip portion with a V-shaped distal end to facilitate efficient and accurate use.

### BACKGROUND OF THE INVENTION

A goniotomy is a surgical procedure primarily used to treat congenital glaucoma. It is caused by a developmental arrest of some of the structures within the anterior (front) segment of the eye. These structures include the iris and the ciliary body, which produces the aqueous fluid needed to maintain the integrity of the eye. These structures do not develop normally in the eyes of patients with isolated congenital glaucoma. Instead, they overlap and block the trabecular meshwork, which is the primary drainage system for the aqueous fluid. As a result of this blockage, the trabecular meshwork itself becomes thicker and the drainage holes within the meshwork are narrowed. These changes lead to an excess of fluid in the eye, which can cause pressure that can damage the internal structures of the eye and cause glaucoma.

The purpose of a goniotomy is to clear the obstruction to aqueous outflow from the eye, which in turn lowers the intraocular pressure (IOP). Lowering the IOP helps to stabilize the enlargement of the cornea and the distension and stretching of the eye that often occur in congenital glaucoma. The size of the eye, however, will not return to normal. Most importantly, once the aqueous outflow improves, damage to the optic nerve is halted or reversed. The patient's visual acuity may improve after surgery.

The goniotomy procedure can restore normal drainage of aqueous humor from the eye by removing a full thickness segment of the trabecular meshwork, thus allowing the aqueous humor to drain through the open area from which the strip of trabecular meshwork has been removed. The goniotomy procedure and certain prior art instruments useable to perform such procedure are described in U.S. Pat. No. 6,979,328, hereby incorporated by reference in its entirety.

At present there remains a need in the art for the development of simple, inexpensive, and accurate instruments useable to perform the procedure of cutting or disrupting the trabecular meshwork in the eye.

### BRIEF SUMMARY OF THE INVENTION

In accordance with the present invention, a micro-surgical instrument is disclosed which is particularly configured to facilitate performing goniotomy such as for treatment of glaucoma. The instrument includes a specifically configured cutting portion which facilitates cutting or disruption of the trabecular meshwork to permit drainage of aqueous humor to enhance the vision of the patient.

In accordance one form of the present invention, the goniotomy surgical instrument includes a hand grip portion having an elongated configuration, including proximal and distal ends. The instrument includes a tip portion extending from the distal end of the hand grip portion, with the tip portion dimensioned to perform the desired cutting or disruption of the trabecular meshwork. The tip portion includes a shank extending from the hand grip portion and a V-shaped distal end having first and second legs. The first leg extends at a first angle from the shank of the tip portion. The second leg extends at a second angle from the first leg. The shank and the first and second legs are substantially coplanar.

In one preferred form of the invention, the first angle is between about 90 and 180 degrees. More preferably, the first angle is about 130 degrees, +/−5 degrees.

In one preferred form of the invention, the second angle is between about 0 and 90 degrees. More preferably, the second angle is about 44 degrees.

In another preferred form of the invention, the shank of the tip portion tapers radially inwardly in a direction moving away from the hand grip portion.

In still another preferred form of the invention, the free end of the second leg is shaped to define a flattened edge region. Preferably, the flattened end region of the free end defines a cutting surface.

According to one preferred form of the invention, at least the second leg of the tip portion has a hollow configuration.

According to another form of the invention, the second leg includes a free end having a convex bottom surface and an oppositely facing, concave top surface.

According to still another form of the invention, at least a free end of the second leg is formed from one of a metal or a polymer.

In yet another form of the invention, at least a free end of the second leg is formed from a substrate with at least one coating deposited thereon. Preferably, the substrate is silicon and the at least one coating deposited thereon is carbon.

In another form of the invention, at least a free end of the second leg is formed from a stamped wire material.

According to still another form of the invention, at least a free of the second leg is formed from an injection molded material.

According to still another form of the invention, at least a free end of the second leg is removably attached to the hand grip portion of the instrument.

In one preferred form of the invention, the hand grip portion of the instrument is one of: a longitudinally vibrating phacoemulsification handpiece; a torsionally-vibrating phacoemulsification handpiece, an elliptically vibrating phacoemulsification handpiece, a phacoemulsification handpiece configured for vibratory movement in three dimensions, a vitrectomy handpiece, a piezo electric handpiece, a solenoid valve handpiece, or a battery powered handpiece. More preferably, the hand grip portion of the instrument is coupled with an irrigation supply source to provide an irrigation fluid through the instrument or around the instrument with an irrigation sleeve coupled or arranged around a portion of the instrument. In one preferred form of the invention, the hand grip portion includes a reservoir of a fluent substance and a pressure activated button for being engaged to release the fluent substance as a jet or reflux from the tip portion of the instrument.

In one preferred form of the invention, the tip portion of the instrument is hollow and defines at least a first aperture or port in the second leg for accommodating flow of a fluent substance through the instrument and out of the first aperture. Preferably, the first aperture in the second leg is centered along a central axis defined by the second leg for accommodating a first flow of a fluent substance through the instrument and out of the first aperture generally along the central axis. Even more preferably, the tip portion defines at least a second aperture extending in a lateral direction relative to the first aperture and located in the second leg for accommodating a second direction of flow of a fluent substance through the instrument that is generally normal to the first direction of flow.

In one form of the invention, the tip portion is selectively retractable into the hand grip portion of the instrument.

In another form of the invention, the first leg has a substantially rectangular cross-sectional shape in a plane that extends in a normal direction relative to a central axis defined by the first leg, and the second leg has a substantially rectangular cross-sectional shape in a plane that extends in a normal direction relative to a central axis defined by the second leg.

According to another form of the invention, the first leg and the second leg define a pair of opposite, planar surfaces.

According to one broad form of the invention, the instrument includes a hand grip portion having an elongated configuration, having proximal and distal ends and a tip portion extending from the distal end of the hand grip portion. The tip portion includes a free end defined by a concave surface that extends from a full thickness portion of the tip portion to a thinner, partially cylindrical flat end surface.

According to another broad form of the invention, the instrument includes a hand grip portion having an elongated configuration, having proximal and distal ends and a tip portion extending from the distal end of the hand grip portion. The tip portion includes a free end defining a central axis and having a convex bottom surface and a concave top surface when viewed in a cross-sectional plane that extends in a normal direction relative to the central axis.

### BRIEF DESCRIPTION OF THE DRAWINGS

In the accompanying drawings forming part of the specification, in which like numerals are employed to designate like parts throughout the same,

FIG. 1 is a left side elevation view of a first embodiment of a surgical instrument according to the present invention;

FIG. 2 is a greatly enlarged, fragmentary view of the circled portion of the instrument of FIG. 1;

FIG. 3 is a greatly enlarged, fragmentary, perspective view taken from above of the operative, tip portion of the instrument shown in FIG. 1, and the instrument in FIG. 3 is rotated 180 degrees from the orientation shown in FIG. 1;

FIG. 4 is a greatly enlarged, fragmentary, perspective view taken from above and from the rear of the operative, tip portion of the instrument shown in FIG. 1;

FIG. 5 is a greatly enlarged, fragmentary view of the circled portion of the instrument of FIG. 4;

FIG. 6 is a greatly enlarged, fragmentary, rear elevation view of a portion of the instrument shown in FIG. 1;

FIG. 7 is a greatly enlarged, fragmentary, perspective view, taken from above and from the rear of the operative, tip portion of another embodiment of an instrument according to the present invention;

FIG. 8 is a greatly enlarged, fragmentary, right side elevation view of a portion of the instrument shown in FIG. 7;

FIG. 9 is a greatly enlarged, fragmentary, top elevation view of a portion of the instrument shown in FIG. 7;

FIG. 10 is a greatly enlarged, fragmentary, perspective view, taken from above and from the rear of the operative, tip portion of another embodiment of an instrument according to the present invention;

FIG. 11 is a greatly enlarged, fragmentary, top elevation view of a portion of the instrument shown in FIG. 10;

FIG. 12 is an enlarged, fragmentary, right side elevation view, of another embodiment of an instrument according to the present invention;

FIG. 13 is an enlarged, fragmentary, right side elevation view, of yet another embodiment of an instrument according to the present invention;

FIG. 14 is an enlarged, fragmentary, right side elevation view, of another embodiment of an instrument according to the present invention;

FIG. 15 is an enlarged, fragmentary, right side elevation view, of yet another embodiment of an instrument according to the present invention;

FIG. 16 is an enlarged, fragmentary, right side elevation view, of another embodiment of an instrument according to the present invention;

FIG. 17 is a diagrammatic, simplified view of the instrument of FIG. 1 in communication with a handpiece and, optionally, a fluid supply source and an irrigation sleeve;

FIG. 18 is a right-side elevation view of another embodiment of a surgical instrument according to the present invention, and FIG. 18 shows the tip portion of the instrument connected to a backflush type irrigating handle with a reservoir of irrigating fluid;

FIG. 19 is a greatly enlarged, left side elevation view of the instrument shown in FIG. 18, and FIG. 19 shows the tip portion of the instrument connected to a backflush type irrigating handle with a reservoir of irrigating fluid and three irrigation fluid paths indicated by arrows;

FIG. 20 is a greatly enlarged, perspective view, taken from above and the right, of the instrument shown in FIG. 18;

FIG. 21 is an enlarged, perspective view, taken from above and the right, of the tip portion of another surgical instrument according to the present invention, and FIG. 21 shows the tip portion only of an instrument for being attached to an irrigating handpiece;

FIG. 22 is a fragmentary, greatly enlarged, perspective view, taken from below and the right, of the tip portion of the instrument in FIG. 21;

FIG. 23 is a top plan view of another surgical instrument according to the present invention, and FIG. 23 shows the tip portion of the instrument retracted within the handle of the instrument;

FIG. 24 is a top plan view of the instrument of FIG. 23, and FIG. 24 shows the tip portion of the instrument extended out of the handle of the instrument;

FIG. 25 is an enlarged, perspective view, taken from above and the left, of just the operative, tip portion of the surgical instrument of FIG. 23, and FIG. 25 shows the tip portion only of an instrument for being attached to a handle or handpiece;

FIG. 26 is a left side elevation view of the tip portion of the instrument in FIG. 25;

FIG. 27 is a rear elevation view of the tip portion of the instrument of FIG. 25;

FIG. 28 is a greatly enlarged, fragmentary, left side elevation view of the tip portion of another surgical instrument according to the present invention, and FIG. 28 shows a gentle, right-angled bent tip portion only of an instrument for being attached to an irrigating handpiece;

FIG. 29 is a greatly enlarged, fragmentary, left side elevation view of the tip portion of another surgical instrument according to the present invention, and FIG. 29 shows a sharp, right-angled bent tip portion only of an instrument for being attached to an irrigating handpiece;

FIG. 30 is a greatly enlarged, fragmentary, left side elevation view of the tip portion of another surgical instrument according to the present invention, and FIG. 30 shows a sharp, downwardly bent tip portion (when the handle is gripped by a user of the instrument) only of an instrument for being attached to an irrigating handpiece;

FIG. 31 is a greatly enlarged, fragmentary, left side elevation view of the tip portion of another surgical instrument according to the present invention, and FIG. 31 shows a sharp, upwardly bent tip portion (when the handle is gripped by a user of the instrument) only of an instrument for being attached to an irrigating handpiece;

FIG. 32 is a greatly enlarged, fragmentary, left side elevation view of the tip portion of another surgical instrument according to the present invention, and FIG. 32 shows a gentle, upwardly bent tip portion (when the handle is gripped by a user of the instrument) only of an instrument for being attached to an irrigating handpiece; and

FIG. 33 is a greatly enlarged, fragmentary, left side elevation view of the tip portion of another surgical instrument according to the present invention, and FIG. 33 shows a gentle, downwardly bent tip portion (when the handle is gripped by a user of the instrument) only of an instrument for being attached to an irrigating handpiece.

### DETAILED DESCRIPTION OF THE PREFERRED EMBODIMENTS

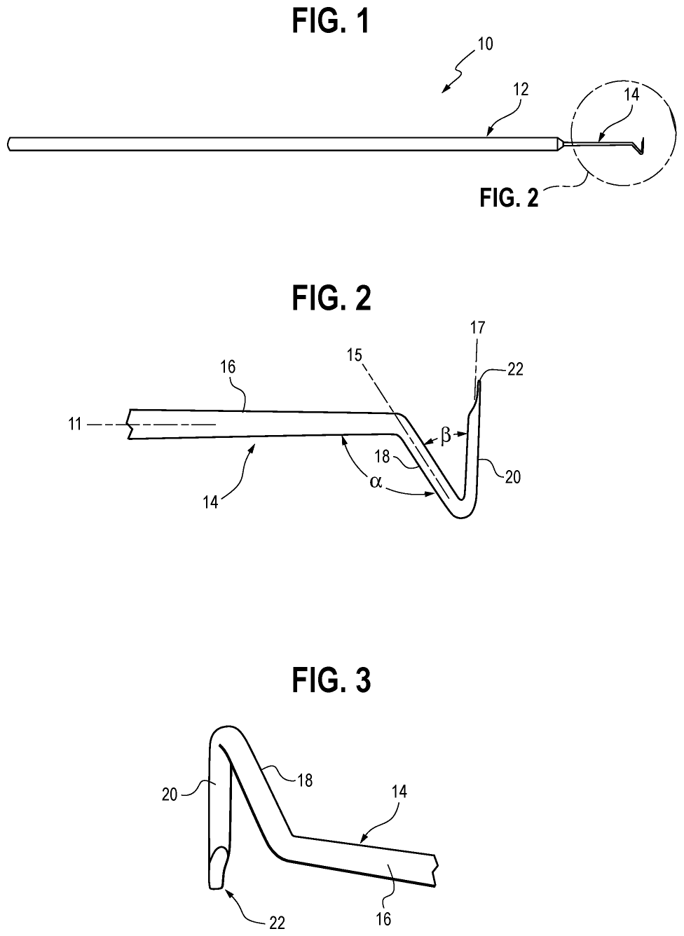

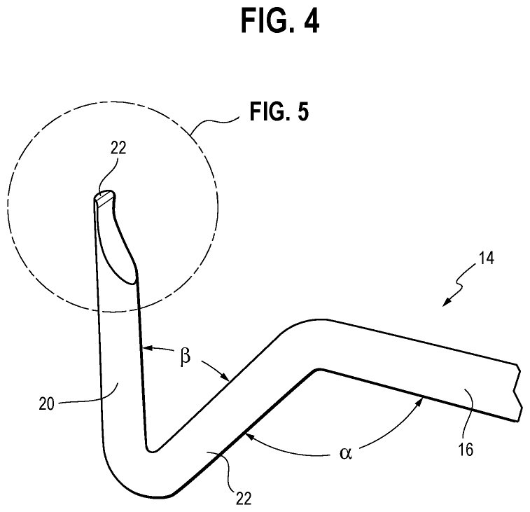

With reference to FIGS. 1-6, in accordance with first illustrated embodiment of a micro-surgical instrument 10 of the present invention is disclosed which is particularly configured to facilitate performing goniotomy such as for treatment of glaucoma. The instrument 10 includes a specifically configured operative, distal portion which facilitates disruption of the trabecular meshwork to permit drainage of aqueous humor to enhance the vision of the patient.

With reference to FIG. 1, the goniotomy surgical instrument 10 includes a handle or hand grip portion 12 for being gripped by a user of the instrument 10 having an elongated configuration, including proximal and distal ends. The instrument includes a tip portion 14 extending from the distal end of the hand grip portion, with the tip portion dimensioned to perform the desired disruption of the trabecular meshwork.

With reference to FIG. 2, the tip portion 14 of the instrument includes a shank 16 extending from the hand grip portion 12, and a V-shaped distal end having a first leg 18 and a second leg 20. As illustrated, the first leg 18 extends at a first angle α from the shank 16 of the tip portion 14, and the second leg 20 extends at a second angle β from the first leg 18, with shank 16 and the first and second legs 18, 20 being substantially coplanar. The shank defines a central, shank axis 11 extending from the handle 12 to the connection with the first leg 18. The first leg 18 defines a central, first leg axis 15. The second leg defines a central, second leg axis 17.

Still referring to FIG. 2, the first angle α of the V-shaped distal end is between about 90 and 180 degrees, that is an obtuse angle, illustrated as being about 130 degrees. In contrast, the second angle β of the V-shaped distal end is between about 0 and 90 degrees, that is an acute angle, shown as being about 44 degrees.

In the present goniotomy surgical instrument 10, the shank 16 of the tip portion 14 tapers inwardly in a direction away from the associated hand grip portion 12. The operative, free end or distal end 22 of the tip portion 14 can be made into a circular open cannula (illustrated in later discussed embodiments), or a solid tubular end as illustrated in FIGS. 1-6. The instrument distal end 22 may be formed or made from metal, a molded material plastic material, or it may be insert-molded from two different materials to form a composite. In one form, the instrument distal end may be stamped from a round wire to achieve its final shape.

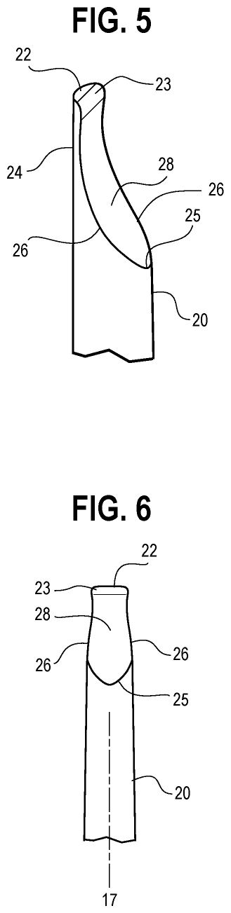

With reference to FIG. 5, the free end 22 of the second leg 20 is shaped to define a flattened edge region or surface that is preferably blunted. A convex, rounded portion 23 leads toward a concave top surface 28 leading rearwardly along the second leg axis 17. The concave surface 28 extends from a distal, thinnest portion and terminates at proximal thickest portion at a central location 25. Opposite the concave surface 28 is a convex or rounded bottom surface 24. A pair of arcuate lateral edges 26 are defined on either side of the axis 17 along the perimeter of the concave surface 28.

A goniotomy surgical instrument 10 in accordance with the present invention includes a hand grip portion 12 which can be provided with either a rounded or a flattened configuration.

The inventors have found that the beveled, dulled, or smoothed edge region of the free end 22 and the concave surface 28 may prevent the tissue of the eye from becoming trapped between irregularities on the surface of the instrument 10, reducing coring and insertion force—improving the cutting capability of the instrument 10.

The inventors have found that the V-shaped tip portion 14 of the instrument 10 advantageously presents the free end 22 in a parallel position for assisting the surgeon to enter into the Schlemm's Canal through a trap entry. The scalloped or spatulated free end 22 has been found to be particularly useful for trap entry and does not require any secondary device to make such a wound for entry. The free end 22 is shaped to rest on the Schlemm's canal and it can be used to fixate before it is used to make an incision in the eye.

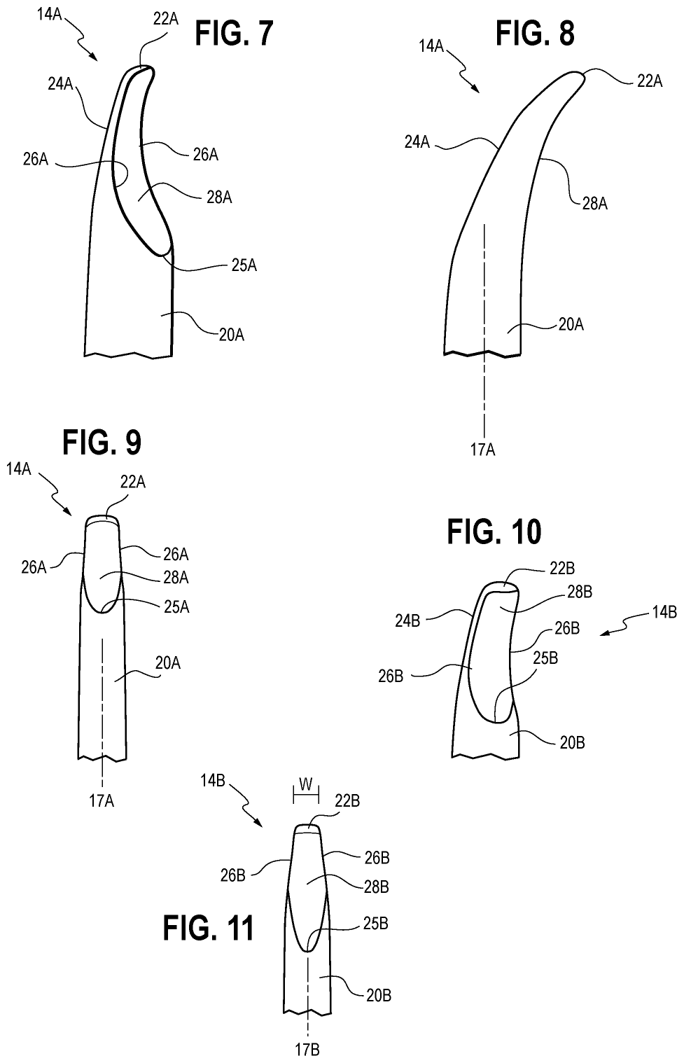

Referring now to FIGS. 7-9, a tip portion of another illustrated embodiment of the instrument of the present invention is designated as 14A. The numbered features or portions of the tip portion 14A are designated generally with the suffix letter “A” and are analogous to features of the aforementioned illustrated embodiment of the instrument 10 that share the same number (without the suffix letter “A”). The instrument possessing the tip portion 14A operates in an identical manner as described in detail above and has the same basic features of an elongate handle (not illustrated), first leg (not illustrated) and second leg 20A together forming a V-shaped tip extending from the handle, and a free end 22A. The tip portion 14A differs from the aforementioned embodiment of the instrument 10 in that the tip portion 14A includes an arcuate or convex curved bottom surface 24A at the free end 22A. The curved bottom surface 24A extends on an opposite surface or portion of the free end 22A as the sloping, concave top surface 28A of the free end 22A. The inventors have found that the tip portion 14A as illustrated having a curved bottom surface 24A extends on an opposite surface or portion of the free end 22A as the sloping, concave top surface 28A of the free end 22A may be ideally suited for following the contour of the shape of the Schlemm's canal.

With reference now to FIGS. 10 and 11, an alternate embodiment of a tip portion 14B of an instrument according to the present invention is illustrated having a narrower distal, free end 22B with the tip portion 14B shown in perspective view and top view. The numbered features or portions of the tip portion 14B are designated generally with the suffix letter “B” and are analogous to features of the aforementioned illustrated embodiment of the instrument 10 that share the same number (without the suffix letter “B”). The instrument possessing the tip portion 14B operates in an identical manner as described in detail above and has the same basic features of an elongate handle (not illustrated), first leg (not illustrated) and second leg 20B together forming a V-shaped tip extending from the handle, and a free end 22B. The free end 22B tapers more drastically than the prior discussed embodiment and has a substantially reduced terminal width “W” (FIG. 11) compared to the full width of the second leg 20B. The free end 22B is further provided with a curved bottom surface 24B extending on an opposite surface or portion of the free end 22B as the sloping, concave top surface 28B of the free end 22B for following the contour of the shape of the Schlemm's canal.

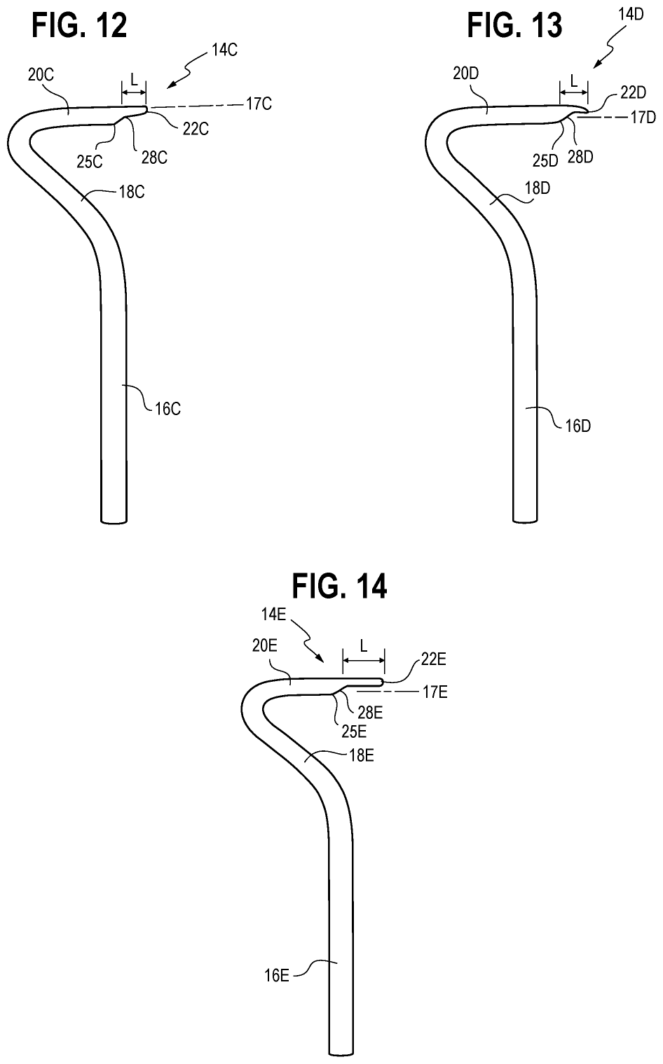

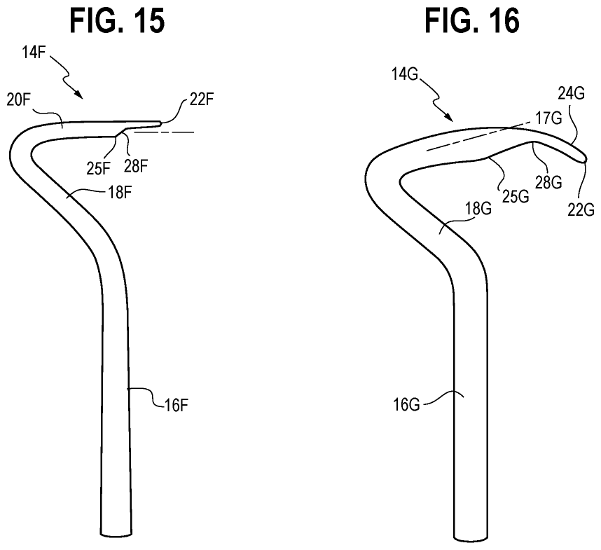

With reference now to FIGS. 12-16, additional embodiments of tip portions of an instrument according to the present invention are illustrated. The numbered features or portions of the tip portions 14B-14G are designated generally with the suffix letters “B-G” and are analogous to features of the aforementioned illustrated embodiment of the instrument 10 that share the same number (without the suffix letters “B-G”). Referring to FIG. 12, the tip portion 14C of the instrument defines a length “L” of the operative, thinned portion of the instrument from the central location 25C to the free end 22C, taken along the axis 17C of the second leg 20C. The thinned portion along the length “L” can be seen to be generally straight, extending along the axis 17C. Referring to FIG. 13, the tip portion 14D of the instrument defines a length “L” of the operative, thinned portion of the instrument from the central location 25D to the free end 22D, taken along the axis 17D of the second leg 20D. Further, the thinned portion of the instrument along the length “L” in FIG. 13 is arcuate, curving back toward the axis 17D. Referring to FIG. 14, the tip portion 14E of the instrument defines a length “L” of the operative, thinned portion of the instrument from the central location 25E to the free end 22E, taken along the axis 17E of the second leg 20E. Further, the thinned portion of the instrument along the length “L” in FIG. 14 is elongate, extending about twice the length, or greater, compared to the prior discussed embodiments along the axis 17E. FIG. 15 shows another embodiment of an elongate thinned portion of the tip 14F of the instrument that extends in parallel to the axis 17F. FIG. 16 shows still another embodiment of an elongate thinned portion of the tip 14G of the instrument that extends in an arcuate manner away from the axis 17G.

In some forms of the instrument, at least the free end may be removable from the remaining portion of the instrument handle or hand grip portion for disposal, replacement, cleaning, or sharpening. In some forms, at least the free end of the instrument can be made with hard plastic or stainless steel or alternative materials conducive to a MIGS (Micro-Incision Glaucoma Surgery) technique. Furthermore, the include the components of the instrument that are made of metal can be made with a metal stamping process, machined, and manufactured or can be hand finished to reach the desired shapes.

In one broad form of the invention, the second leg and free end thereof may have any desired shape so long as the first and second legs are arranged in the desired V-shaped tip extending from the handle as shown for use in a goniotomy procedure.

The inventors have further found that the instruments disclosed herein are suitable for engaging the Schlemm's canal in 360 degrees without requiring the user to flip the instrument like conventional hooks, or without requiring multiple instruments (i.e., a specific left and/or right hook).

It will be understood that the instrument may be formed in a variety of sizes for small incision glaucoma surgery or regular glaucoma surgery.

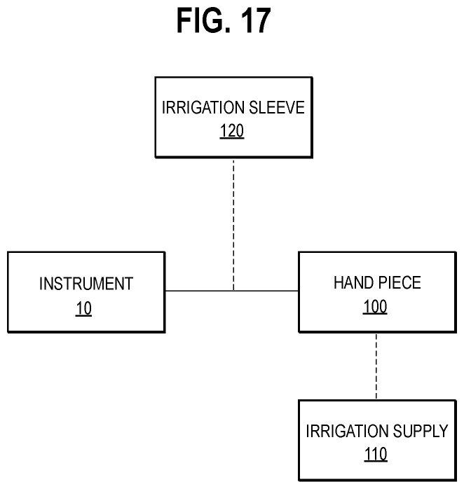

With reference to FIG. 17, in another broad form of the invention, the embodiments of the instruments disclosed herein are capable of being attached or otherwise coupled with a movable hand grip portion in the form of a handpiece 100 to assist in performing the goniotomy procedure. The handpiece 100 may be, for example, a longitudinally vibrating phacoemulsification handpiece, a torsionally-vibrating phacoemulsification handpiece, an elliptically vibrating phacoemulsification handpiece, a phacoemulsification handpiece configured for vibratory movement in three dimensions, a vitrectomy handpiece, a piezo electric handpiece, an ultrasound handpiece, a solenoid valve handpiece, pneumatic, or a battery powered handpiece. Other vibratory handpieces may be used with the instruments 10 disclosed herein.

The handpiece 100 may be developed for the treatment of Open Angle Glaucoma, specifically at the Juxtacanalicular Space (JCS), but not limited to, or based on, combination treatments including vibrating, pulsating, oscillating, Guillotine, Piezo , Radiofrequency (RF), Neodymium-doped yttrium aluminum garnet (Nd:YAG) Laser platforms with specially designed tips and/or Laser probes.

The proximal end or portion of the instrument 10 would not function as a hand grip 12, per se, when incorporated into a handpiece 100, and may be removably or non-removably coupled with the handpiece 100, such as by mating threads, luer lock, force fit, snap-fit, etc.

Still referring to FIG. 17, in some applications the goniotomy surgical instruments disclosed herein may be coupled with an irrigation fluid supply source 110 to provide an irrigation fluid either (i) through one or more cannulas in the instruments, or (ii) around the exterior surface of the tip portion of the instruments when coupled with an irrigation sleeve 120 arranged around a portion of the distal end of the instrument 10.

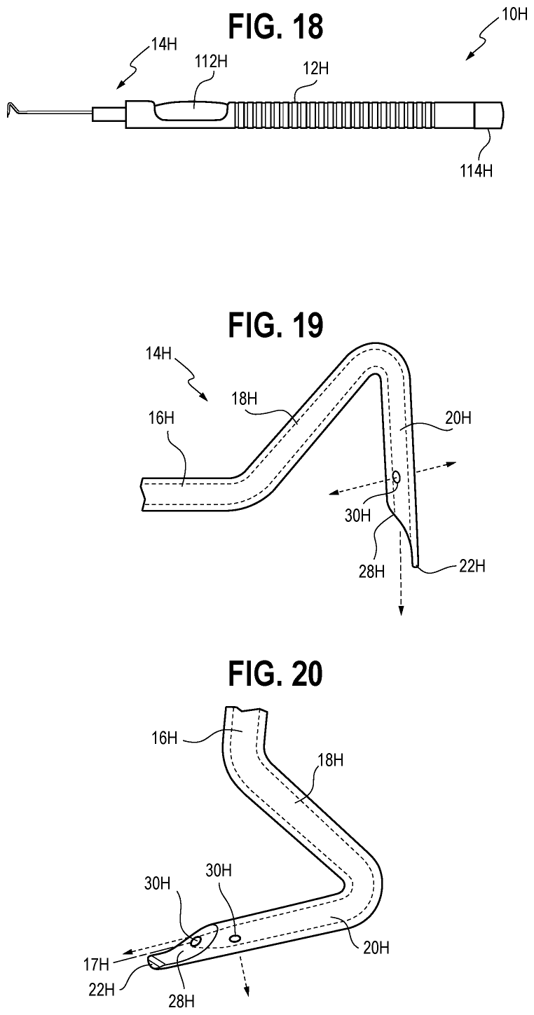

One such irrigating embodiment of the instrument according to the present invention is illustrated in FIGS. 18-20, wherein the instrument 10H includes a handle 12H containing a stored amount of fluent solution (such as BSS) and a pressure activated chamber that is capable of flushing or providing a jet or reflux stream of stored fluent solution through the distal tip portion 14H of the instrument.

In this illustrated embodiment of the instrument 10H, the numbered features or portions of the tip portion 14H are designated generally with the suffix letter “H” and are analogous to features of the aforementioned illustrated embodiment of the instrument 10 that share the same number (without the suffix letter “H”). The instrument possessing the tip portion 14H operates in an identical manner as described in detail above and has the same basic features of an elongate handle 12H, first leg 18H and second leg 20K together forming a V-shaped tip portion 14H extending from the handle 12H, and a spatulated free end 22H having an arcuate, concave surface 28H.

The instrument 10H differs from the prior discussed non-irrigating embodiments in that the tip portion 14H is provided with one or more apertures or ports 30H connecting to a central lumen to facilitate flushing of the tissues of the eye during use of the instrument. In one preferred form, the tip portion 14H includes a first, central or longitudinal aperture 30H for flushing fluid along a central axis 17H of the second leg 20H and a two side or lateral (left and right) apertures 30H for flushing fluid in opposite direction normal or orthogonal to the central axis of the second leg 20H. In other embodiments (not illustrated), the tip portion 14H may be provided with only a single longitudinal aperture 30H, or two apertures 30H. The inventors believe that the irrigation of the instrument 10H may be particularly suitable for eliminating or at least reducing the adhesion of tissue on the Schlemm's canal during surgery and for massaging the tissues of the canal.

In some applications, the inventors have found that the irrigating forms of the instrument 10H may be used for the revision or repair of the operative wound of the anterior segment of any type, early or late, major, or minor, procedure. The dull distal end of the instrument tip portion can be utilized to internally reopen and repair a scarred sclerotomy, as well as to revise, irrigate, and refill the conjunctival filtering bleb.

In some applications, the inventors have found that the instrument 10 with the V-shaped distal configuration, having first and second legs, is capable of engaging, retracting, and slicing the fibrous anterior and posterior synechiae tissues. A clockwise or counterclockwise 360 degrees careful movement rotation of the V-shaped configuration of the instrument is capable of freeing the iris and/or pupillary margin from membranes and/or attachments to the posterior corneal endothelial layer, and/or the anterior lens capsule.

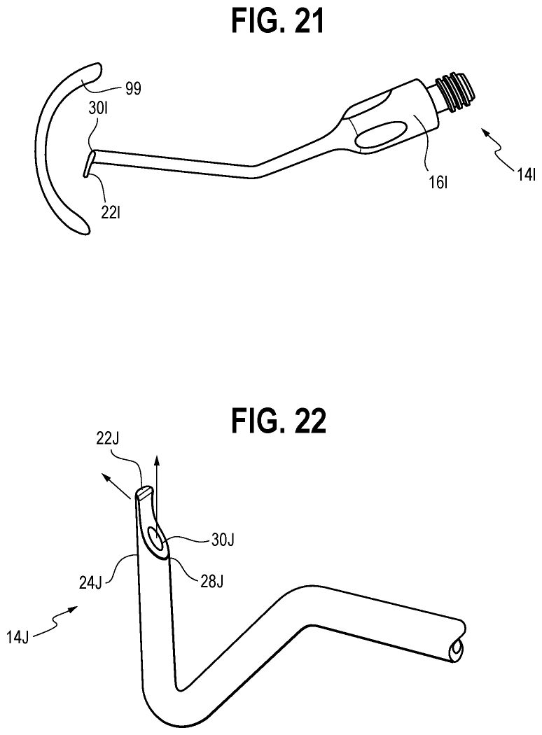

With reference now to FIG. 21, another irrigating embodiment of the instrument according to the present invention is illustrated, wherein the instrument includes a tip portion 14I for being threaded onto a handle (not illustrated) in communication with a supply of a fluent solution (such as BSS).

In this illustrated embodiment of the instrument, the numbered features, or portions of the tip portion 14I are designated generally with the suffix letter “I” and are analogous to features of the aforementioned illustrated embodiment of the instrument 10 that share the same number (without the suffix letter “I”). The instrument possessing the tip portion 14I operates in an identical manner as described in detail above and has the same basic features of an elongate handle or handpiece and a tip portion 14I extending from the handpiece, and a spatulated free end 221 having an arcuate, concave surface (not visible in FIG. 21). The tip portion 14I is provided with one aperture 301 connecting to a central lumen to facilitate flushing of the tissues of the eye during use of the instrument. The Schlemm's canal is represented diagrammatically by the numeral 99 in FIG. 21.

With reference now to FIG. 22, another irrigating embodiment of the instrument according to the present invention is illustrated, wherein the instrument includes a tip portion 14J for being threaded onto a handle (not illustrated) in communication with a supply of a fluent solution (such as BSS).

In this illustrated embodiment of the instrument, the numbered features, or portions of the tip portion 14J are designated generally with the suffix letter “J” and are analogous to features of the aforementioned illustrated embodiment of the instrument 10 that share the same number (without the suffix letter “J”). The instrument possessing the tip portion 14J operates in an identical manner as described in detail above, with respect to the tip portion 14I, and has the same basic features of an elongate handle or handpiece and a tip portion 14Jextending from the handpiece, and a spatulated free end 22J having an arcuate, concave surface 28J. The tip portion 14J is provided with a first longitudinal aperture 30J in the concave surface 28J and an orthogonally spaced second aperture 30J in the bottom surface 24J, each connecting to a central lumen to facilitate flushing of the tissues of the eye during use of the instrument.

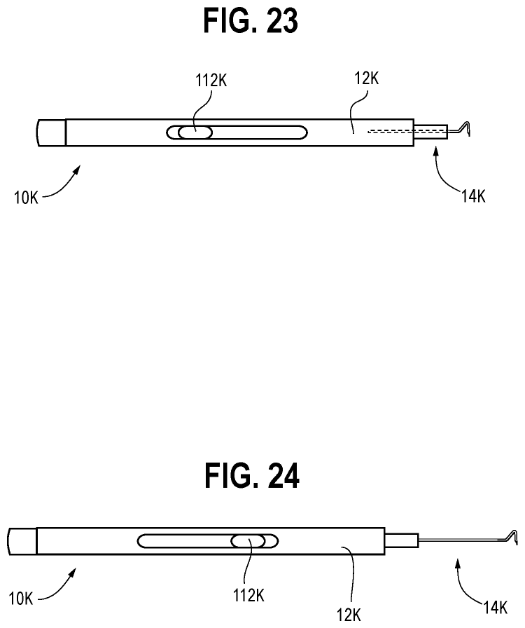

With reference now to FIGS. 23-27, another embodiment of an instrument according to the present invention is illustrated, wherein the instrument 10K includes a tip portion 14K extending outwardly from a handpiece or handle 12K.

In this illustrated embodiment of the instrument 10K, the numbered features or portions of the instrument 10K are designated generally with the suffix letter “K” and are analogous to features of the aforementioned illustrated embodiment of the instrument 10 that share the same number (without the suffix letter “K”). The instrument 10K operates in an identical manner as described in detail above with respect to the first illustrated embodiment of the invention, and it has the same basic features of a shank portion 16K, a first leg 18K, and second leg 20K together forming a V-shaped tip portion 14K extending from the handle 12K, and a spatulated free end 22K having an arcuate, concave surface 28K.

As can be seen in FIGS. 23 and 24, the tip portion 14K is selectively retractable within the handle 12K with the actuation of a sliding button 112K.

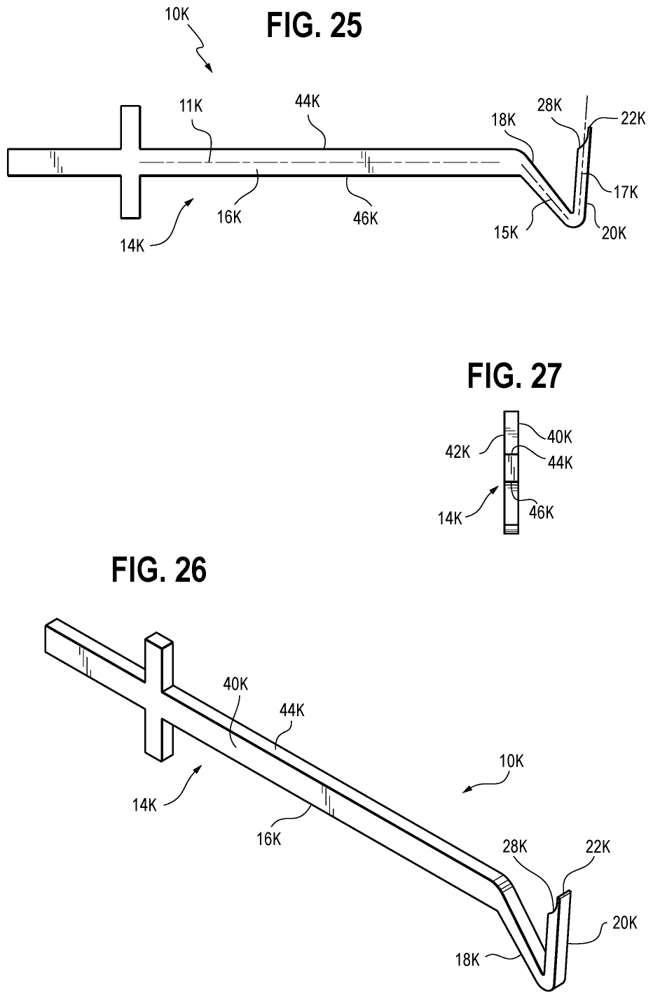

With reference now to FIGS. 25-27, the tip portion 14K of the instrument 10K has a somewhat planar configuration. The tip portion 14K of the instrument 10K is preferably formed by chemical vapor deposition of carbon on a silicon wafer substrate for improved manufacturing tolerances and manufacturing costs. The tip portion 14K may alternatively be formed or made from metal, a molded plastic material, or it may be insert-molded from two different materials to form a composite. In one form, the instrument tip portion 14K may be stamped or cut from a plate or wire to achieve its final shape.

With reference to FIG. 27, it can be seen that the cross-section of the shank 16K, first and second legs 18K and 20K, which are substantially uniform, have the form of a rectangle, defining a first pair of opposite, lateral sides 40K and 42K, and a pair of opposite, top and bottom sides 44K and 46K. The concave surface 28K further has the form of a curved plane.

In one presently preferred form of the instrument 10K, the width of the tip portion 14K, between the lateral sides 40K and 42K, as between about 0.2 and 0.5 mm, and is more preferably about 0.3 mm.

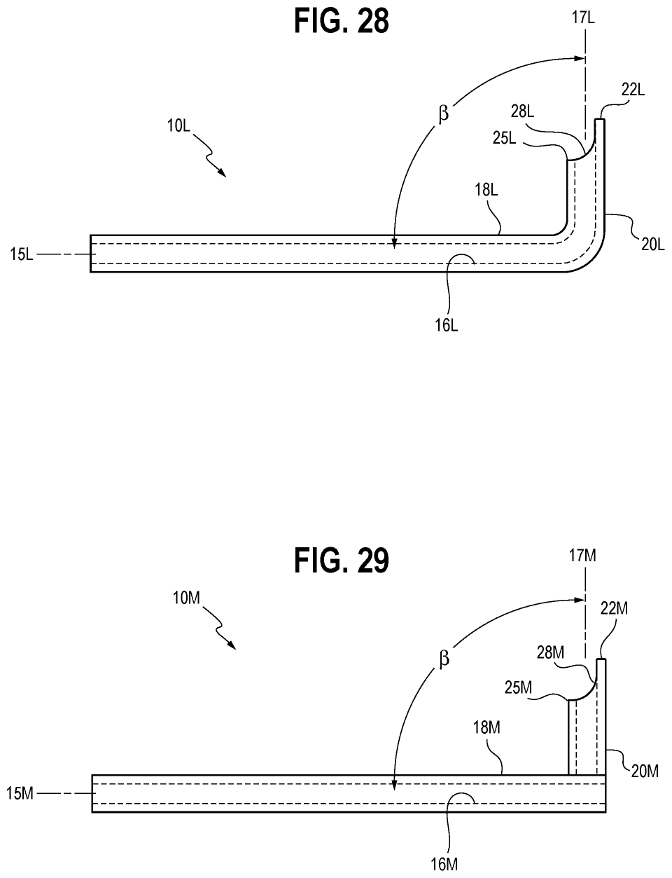

With reference now to FIG. 28, a tip portion of another illustrated embodiment of an instrument of the present invention is designated as 10L. The numbered features or portions of the instrument 10L are designated generally with the suffix letter “L” and are analogous to features of the aforementioned illustrated embodiment of the instrument 10 that share the same number (without the suffix letter “L”). The instrument 10L operates in an identical manner as described in detail above and has the same basic features of an elongate handle (not illustrated), first leg 18L and second leg 20L together forming an L-shaped tip extending from the distal end of the handle, and a free end 22L. The tip portion of the instrument 10L differs from the aforementioned embodiment of the instrument 10 in that the first leg 18L of the tip portion has a central axis 15L that is coaxial with the handle, and the second leg 20L defines a central axis 17L that is bent at an angle β of about ninety degrees relative to the axis 15L. The first and second legs 18L and 20L may be provided with an irrigation passage or through passage 16L that may terminate in one or more ports in the first leg 18L and/or second leg 20L as discussed above with respect to the other embodiments. The second leg 20L includes an arcuate or convex curved bottom surface at the free end 22L and an opposite surface or portion of the free end 22L as the sloping, concave top surface 28L with a trailing edge 25L. When the instrument 10L is gripped by the handle portion, which is held level, then the second leg 20L will be oriented upwardly. The instrument 10L further has a gentle, convex bend between the first leg 18L and the second leg 20L for improved for improved safety and manufacturability.

With reference now to FIG. 29, a tip portion of another illustrated embodiment of an instrument of the present invention is designated as 10M. The numbered features or portions of the instrument 10M are designated generally with the suffix letter “M” and are analogous to features of the aforementioned illustrated embodiment of the instrument 10 that share the same number (without the suffix letter “M”). The instrument 10M operates in an identical manner as described in detail above and has the same basic features of an elongate handle (not illustrated), first leg 18M and second leg 20M together forming an L-shaped tip portion extending from the distal end of the handle, and a cantilevered, free end 22M. The tip portion of the instrument 10M differs from the aforementioned embodiment of the instrument 10 in that the first leg 18M of the tip portion has a central axis 15M that is coaxial with the handle, and the second leg 20M defines a central axis 17M that is bent at an angle β of about ninety degrees relative to the axis 15M. The first and second legs 18M and 20M may be provided with an irrigation passage or through passage 16M that may terminate in one or more ports in the first leg 18M and/or second leg 20M as discussed above with respect to the other embodiments. The second leg 20M includes an arcuate or convex curved bottom surface at the free end 22M and an opposite surface or portion of the free end 22M as the sloping, concave top surface 28M with a trailing edge 25M. When the instrument 10M is gripped by the handle portion, which is held level, then the second leg 20M will be oriented upwardly. The instrument 10M further has a sharp, abrupt bend between the first leg 18M and the second leg 20M for improved manufacturability.

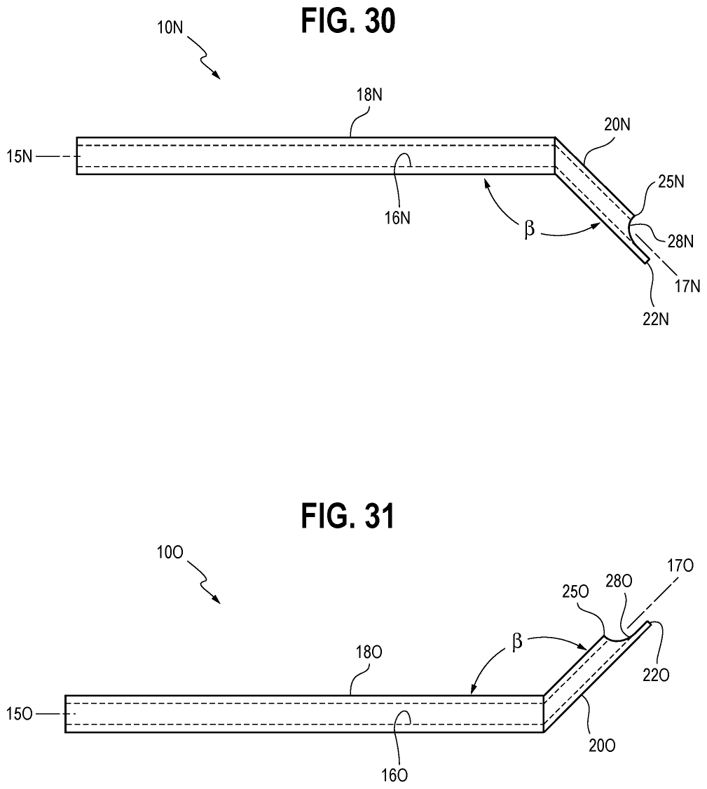

With reference now to FIG. 30, a tip portion of another illustrated embodiment of an instrument of the present invention is designated as 10N. The numbered features or portions of the instrument 10N are designated generally with the suffix letter “N” and are analogous to features of the aforementioned illustrated embodiment of the instrument 10 that share the same number (without the suffix letter “N”). The instrument 10N operates in an identical manner as described in detail above and has the same basic features of an elongate handle (not illustrated), first leg 18N and second leg 20N together forming a bent tip portion extending from the distal end of the handle, and a cantilevered, free end 22N. The tip portion of the instrument 10N differs from the aforementioned embodiment of the instrument 10 in that the first leg 18N of the tip portion has a central axis 15N that is coaxial with the handle, and the second leg 20N defines a central axis 17N that is bent at an angle β of about 135 degrees relative to the axis 15N. The first and second legs 18N and 20N may be provided with an irrigation passage or through passage 16N that may terminate in one or more ports in the first leg 18N and/or second leg 20N as discussed above with respect to the other embodiments. The second leg 20N includes an arcuate or convex curved bottom surface at the free end 22N and an opposite surface or portion of the free end 22N as the sloping, concave top surface 28N with a trailing edge 25N. When the instrument 10N is gripped by the handle portion, which is held level, then the second leg 20N will be oriented downwardly. The instrument 10N further has a sharp, abrupt bend between the first leg 18N and the second leg 20N for improved manufacturability.

With reference now to FIG. 31, a tip portion of another illustrated embodiment of an instrument of the present invention is designated as 10O. The numbered features or portions of the instrument 10O are designated generally with the suffix letter “O” and are analogous to features of the aforementioned illustrated embodiment of the instrument 10 that share the same number (without the suffix letter “O”). The instrument 10O operates in an identical manner as described in detail above and has the same basic features of an elongate handle (not illustrated), first leg 18O and second leg 20O together forming a bent tip portion extending from the distal end of the handle, and a cantilevered, free end 22O. The tip portion of the instrument 10O differs from the aforementioned embodiment of the instrument 10 in that the first leg 18O of the tip portion has a central axis 15O that is coaxial with the handle, and the second leg 20O defines a central axis 17O that is bent at an angle β of about 135 degrees relative to the axis 15O. The first and second legs 18O and 20O may be provided with an irrigation passage or through passage 16O that may terminate in one or more ports in the first leg 18O and/or second leg 20O as discussed above with respect to the other embodiments. The second leg 20O includes an arcuate or convex curved bottom surface at the free end 22O and an opposite surface or portion of the free end 22O as the sloping, concave top surface 28O with a trailing edge 25O. When the instrument 10O is gripped by the handle portion, which is held level, then the second leg 20O will be oriented upwardly. The instrument 10O further has a sharp, abrupt bend or transition between the first leg 18O and the second leg 20O for improved manufacturability.

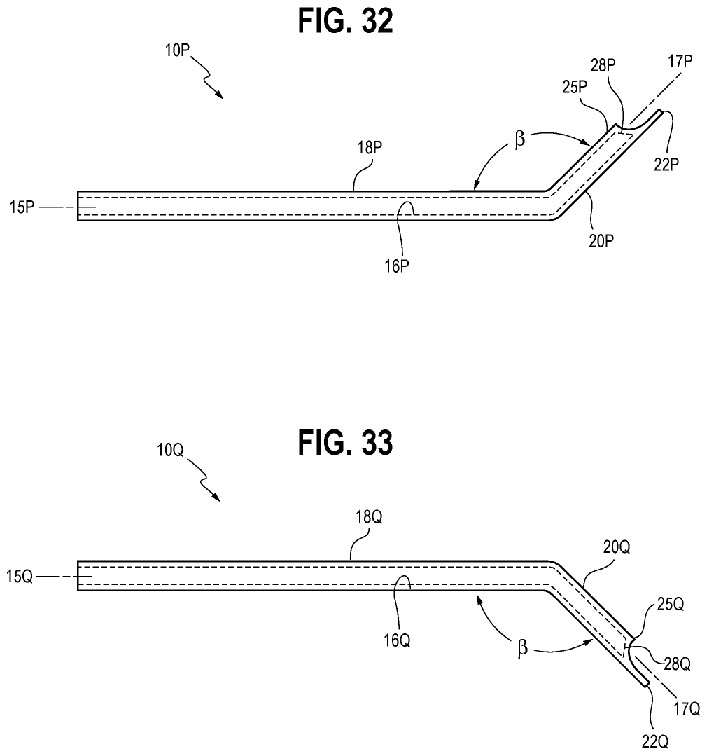

With reference now to FIG. 32, a tip portion of another illustrated embodiment of an instrument of the present invention is designated as 10P. The numbered features or portions of the instrument 10P are designated generally with the suffix letter “P” and are analogous to features of the aforementioned illustrated embodiment of the instrument 10 that share the same number (without the suffix letter “P”). The instrument 10P operates in an identical manner as described in detail above and has the same basic features of an elongate handle (not illustrated), first leg 18P and second leg 20P together forming a bent tip portion extending from the distal end of the handle, and a cantilevered, free end 22P. The tip portion of the instrument 10P differs from the aforementioned embodiment of the instrument 10 in that the first leg 18P of the tip portion has a central axis 15P that is coaxial with the handle, and the second leg 20P defines a central axis 17P that is bent at an angle β of about 135 degrees relative to the axis 15P. The first and second legs 18P and 20P may be provided with an irrigation passage or through passage 16P that may terminate in one or more ports in the first leg 18P and/or second leg 20P as discussed above with respect to the other embodiments. The second leg 20P includes an arcuate or convex curved bottom surface at the free end 22P and an opposite surface or portion of the free end 22P as the sloping, concave top surface 28P with a trailing edge 25P. When the instrument 10P is gripped by the handle portion, which is held level, then the second leg 20P will be oriented upwardly. The instrument 10P further has a gentle, arcuate bend or transition between the first leg 18P and the second leg 20P for improved safety and manufacturability.

With reference now to FIG. 33, a tip portion of another illustrated embodiment of an instrument of the present invention is designated as 10Q. The numbered features or portions of the instrument 10Q are designated generally with the suffix letter “Q” and are analogous to features of the aforementioned illustrated embodiment of the instrument 10 that share the same number (without the suffix letter “Q”). The instrument 10Q operates in an identical manner as described in detail above and has the same basic features of an elongate handle (not illustrated), first leg 18Q and second leg 20Q together forming a bent tip portion extending from the distal end of the handle, and a cantilevered, free end 22Q. The tip portion of the instrument 10Q differs from the aforementioned embodiment of the instrument 10 in that the first leg 18Q of the tip portion has a central axis 15Q that is coaxial with the handle, and the second leg 20Q defines a central axis 17Q that is bent at an angle β of about 135 degrees relative to the axis 15Q. The first and second legs 18Q and 20Q may be provided with an irrigation passage or through passage 16Q that may terminate in one or more ports in the first leg 18Q and/or second leg 20Q as discussed above with respect to the other embodiments. The second leg 20Q includes an arcuate or convex curved bottom surface at the free end 22Q and an opposite surface or portion of the free end 22Q as the sloping, concave top surface 28Q with a trailing edge 25Q. When the instrument 10Q is gripped by the handle portion, which is held level, then the second leg 20Q will be oriented downwardly. The instrument 10Q further has a gentle, arcuate bend or transition between the first leg 18Q and the second leg 20Q for improved safety and manufacturability.

Various modifications and alterations to this invention will become apparent to those skilled in the art without departing from the scope and spirit of this invention. Illustrative embodiments and examples are provided as examples only and are not intended to limit the broadest scope of the present invention.

1. A goniotomy surgical instrument comprising:
a hand grip portion having an elongated configuration, having proximal and distal ends; and
a tip portion extending from the distal end of said hand grip portion, said tip portion including a shank extending from said hand grip portion, and a V-shaped distal end having first and second legs, said first leg extending at a first angle from said shank of said tip portion, and said second leg extending at a second angle from said first leg, said shank and said first and second legs being coplanar.

2. The goniotomy surgical instrument in accordance with claim 1, wherein said first angle is between about 90 and 180 degrees.

3. (canceled)

4. The goniotomy surgical instrument in accordance with claim 2, wherein said second angle is between about 0 and 90 degrees.

5. (canceled)

6. The goniotomy surgical instrument in accordance with claim 1, wherein said shank of said tip portion tapers inwardly in a direction away from said hand grip portion.

7. The goniotomy surgical instrument in accordance with claim 1, wherein a free end of said second leg is shaped to define a flattened edge region.

8. The goniotomy surgical instrument in accordance with claim 7, wherein said flattened end region of said free end defines a cutting surface.

9. The goniotomy surgical instrument in accordance with claim 1, wherein at least said second leg of said tip portion has a hollow configuration.

10. The goniotomy surgical instrument in accordance with claim 1, wherein said second leg includes a free end having a convex bottom surface and a concave top surface.

11. (canceled)

12. The goniotomy surgical instrument in accordance with claim 1, wherein at least a free end of said second leg is formed from a substrate with at least one coating deposited thereon.

13. The goniotomy surgical instrument in accordance with claim 12 wherein said substrate is silicon and said at least one coating deposited thereon is carbon.

14. (canceled)

15. (canceled)

16. (canceled)

17. The goniotomy surgical instrument in accordance with claim 1, wherein said hand grip portion is one of: a longitudinally-vibrating phacoemulsification handpiece; a torsionally-vibrating phacoemulsification handpiece, an elliptically-vibrating phacoemulsification handpiece, a phacoemulsification handpiece configured for vibratory movement in three dimensions, a vitrectomy handpiece, a piezo electric handpiece, a solenoid valve handpiece, or a battery powered handpiece.

18. The goniotomy surgical instrument in accordance with claim 17, wherein said hand grip portion is coupled with an irrigation supply source to provide an irrigation fluid through said instrument or around said instrument with an irrigation sleeve arranged around said instrument.

19. The goniotomy surgical instrument in accordance with claim 18, wherein said hand grip portion includes a reservoir of a fluent substance and a pressure activated button for being engaged to release said fluent substance from said tip portion of said instrument.

20. The goniotomy surgical instrument in accordance with claim 1, wherein said tip portion of said instrument is hollow and defines at least a first aperture in said second leg for accommodating flow of a fluent substance through said instrument and out of said aperture.

21. The goniotomy surgical instrument in accordance with claim 20, wherein said first aperture in said second leg is centered along a central axis defined by said second leg for accommodating a first flow of a fluent substance through said instrument and out of said first aperture along said central axis.

22. The goniotomy surgical instrument in accordance with claim 21, wherein said tip portion defines at least a second aperture extending in a lateral direction relative to said first aperture and located in said second leg for accommodating a second flow of a fluent substance through said instrument generally normal to said first flow.

23. The goniotomy surgical instrument in accordance with claim 1, wherein said tip portion is selectively retractable into said hand grip portion of said instrument.

24. The goniotomy surgical instrument in accordance with claim 1 wherein said first leg has a substantially rectangular cross-sectional shape in a plane that extends in a normal direction relative to a central axis defined by said first leg, and said second leg has a substantially rectangular cross-sectional shape in a plane that extends in a normal direction relative to a central axis defined by said second leg.

25. The goniotomy surgical instrument in accordance with claim 1 wherein said first leg and said second leg define a pair of opposite, planar surfaces.

26. A goniotomy surgical instrument, comprising:
a hand grip portion having an elongated configuration, having proximal and distal ends; and
a tip portion extending from the distal end of said hand grip portion,
said tip portion including a free end defined by a concave surface that extends from a full thickness portion of the tip portion to a thinner, partially cylindrical flat end surface.

27. A goniotomy surgical instrument, comprising:
a hand grip portion having an elongated configuration, having proximal and distal ends; and
a tip portion extending from the distal end of said hand grip portion,
said tip portion including a free end defining a central axis and having a convex bottom surface and a concave top surface, when viewed in a cross-sectional plane that extends in a normal direction relative to said central axis.

28. The goniotomy surgical instrument in accordance with claim 27 further comprising a first leg defining a central axis extending straight from said hand grip portion and a second leg defining said free end extending at an angle relative to said central axis of between about 90 degrees and about 140 degrees.

29.-56. (canceled)
